(19) United States
(12) Patent Application Publication
(10) Pub. No.: US 2020/0288965 A1
(43) Pub. Date: Sep. 17, 2020

(54) SYSTEM AND METHOD FOR ENHANCED IMAGING OF BIOLOGICAL TISSUE

(71) Applicant: SPRING BIOMED VISION LTD., Haifa (IL)

(72) Inventors: Avihu Meir GAMLIEL, Pardes-Hana (IL); Noam ALLON, Haifa (IL)

(21) Appl. No.: 16/814,022

(22) Filed: Mar. 10, 2020

Related U.S. Application Data
(60) Provisional application No. 62/816,343, filed on Mar. 11, 2019.
(60) Provisional application No. 62/866,201, filed on Jun. 25, 2019.

(51) Int. Cl. A61B 3/00; A61B 3/12; A61B 3/14; H04N 9/04; G06T 5/00; G06T 5/50; G06T 7/00; G06T 7/11

(52) U.S. Cl. CPC A61B 3/0025; A61B 3/1241; A61B 3/14; H04N 9/04551; G06T 5/008; G06T 2207/30101; G06T 7/0014; G06T 7/11; G06T 2207/10152; G06T 2207/10024; G06T 2207/30041; G06T 5/50

(57) ABSTRACT

A system and method are presented for use in angiographic imaging. The system comprises: a light source unit, and at least one imaging unit comprising a detector array, wherein the detector array comprises at least first and second types of detector cells having corresponding first and second different spectral response functions defining respectively first and second spectral peaks; and the light source is unit configured for emitting light formed of at least first and second discrete wavelength ranges which are selected to be aligned with said first and second spectral peaks of said first and second types of detector cells.

### TECHNOLOGICAL FIELD

The present invention relates to techniques for enhanced imaging of biological tissue and specifically relates to techniques for imaging blood containing tissue for analyzing biological parameters.

### BACKGROUND

Imaging of biological tissues provides important data for physicians in various applications. Angiography is a technique allowing in vivo imaging of blood vessels. This technique can be used in diagnostics of medical conditions and as assisting tool in different medical operations.

The current techniques for angiography utilize administering a radio-opaque contrast agent into the blood of a living subject. This is followed by acquisition of desired images in X-ray wavelength range to provide clear imaging of the blood vessels over the background of biological tissue.

To avoid the use of X-ray radiation, several imaging techniques have been proposed, using images taken following administration of a fluorescent agent (e.g., sodium fluorescein or indocyanine green) and selected illumination to provide fluorescent response in suitable wavelength ranges from the illuminated tissue. Such imaging techniques can provide efficient angiography of various regions of the body including, e.g. retina, sclera, as well as or mucosa tissues, such as the gastrointestinal luminal walls.

Additional techniques enable full-optical angiography, thus avoiding the need for administration of any material to the blood stream. Generally, visible-light color images provide insufficient contrast to clearly discern smaller blood vessels. However, processing of different images collected in selected wavelength ranges (colors) may provide increased contrast for blood vessels over the tissue background. In some cases, “red-free” images (e.g., images acquired where the camera lens is functionally associated with a green filter to prevent red light from being gathered) provided improved contrast over natural color images.

### GENERAL DESCRIPTION

There is a need in the art for a novel technique enabling non-invasive, efficient angiographic imaging that is operable without the need for administration of contrast enhancing agents to patient's blood stream.

The present invention utilizes optical imaging of a region of interest (e.g. retina, sclera, gastrointestinal luminal wall etc.) under selected illumination and acquisition conditions to provide image data having high contrast with respect to blood vessels. The present technique enables imaging of biological tissue while collecting image data with increased contrast of blood vessels over surrounding tissue, and omitting the need for registration processing required for combining images taken at different times and/or by different imaging arrangements.

The present invention may also overcome registration issues rising from the need to apply processing to two or more images. Generally, according to some embodiments, the present technique utilizes concurrent illumination and image acquisition. Such that image acquisition is performed while the region of interest is illumination with the selected illumination conditions as indicated further below. Additionally, according to some embodiments, the present technique may utilize a single detector array, e.g. array having detector cells for collecting different colors, for collecting the image data. Such concurrent illumination and image collection with a single detector array may be used to omit the need for complicated image registration and processing.

More specifically, the present invention provides a system for use in imaging of biological tissue, and preferably for use in enhanced imaging of tissue containing blood vessels. The system comprises an imaging unit and light source unit and may also comprise or be associated with a processing unit.

The imaging unit includes a detector array which comprises an arrangement of plurality of detector cells, including detector cells of two or more different types arranged in a predetermined array (two-dimensional array). The different types of detector cells differ from one another in their spectral response functions, i.e. the sensitivity of the detector cells to light of different wavelengths. Generally, the different types of detector cells are arranged within the detector array in interlaced arrangement. Thus, output image data collected by one type of detector cells provides an image of the field of view using certain wavelength range (corresponding with spectral response of the detector cells). Images collected by each of the different types of detector cells are associated with a common field of view, thereby not requiring additional registration processing.

The detector array is typically associated/equipped with an optical lens arrangement. The optical lens arrangement is configured for operating at visible light and possibly also at near-visible wavelength range and providing imaging of selected field(s) of view onto the detector array.

Generally, color detector arrays typically used in conventional camera units, include three different types of detector cells configured for collection of light in different colors, such as red, green and blue (RGB). It should be noted that such variation in spectral response function may be provided by wavelength selective filter of the detector cells, such as in a Bayer filter. The spectral response functions of each type of detector cells have peak response at certain wavelengths, typically providing global maximum of the response function. For example, the spectral response function of the detector cells of a first type has a peak response at a wavelength around 600-700 nm, and that of a second type of detector cells has a peak response at a wavelength around 420-480 nm. Considering the example of the detectors cells configured for collecting light of the primary colors (RGB), a response function of third type detector cells has peak response around 500-550 nm.

The light source unit is configured to provide illumination of at least two different wavelength ranges, aligned with the wavelengths of peak responses of corresponding at least two different types of detector cells. More specifically, illumination of the first wavelength range comprises wavelengths corresponding to the peak response of the detector cells of the first type, and illumination of the second wavelength range comprises wavelengths corresponding to the peak response of the second type detector cells. To this end, the light source unit may comprise at least two light sources producing relatively narrow bandwidth of illumination (e.g. LED light sources) in the at least two different selected wavelength ranges, respectively.

The imaging system is configured for use in imaging of biological tissue under illumination of two or more discrete wavelength ranges to provide image data having two or more wavelength components. The use of image data pieces indicative of the different wavelength ranges enables processing of the image data and generating enhanced image with high contrast of blood vessels with respect to surrounding tissue. To this end, the term two or more discrete wavelength ranges indicates that the illumination has at least one minima of light intensity for a certain wavelength between said two or more wavelength ranges (accordingly said two or more wavelength ranges do not fully cover the visible spectrum).

Thus, according to a broad aspect, the present invention provides a system comprising: a light source unit, and at least one imaging unit comprising a detector array, wherein the detector array comprises at least first and second types of detector cells having corresponding first and second different spectral response functions defining respectively first and second spectral peaks; and a light source unit is configured for emitting light forming illumination including at least first and second discrete wavelength ranges, said at least first and second discrete wavelength ranges being aligned with said first and second spectral peaks of said first and second types of detector cells.

According to some embodiments, the detector array may comprise a wavelength selective filter array filtering collected light and defining at least a portion of the first and second spectral response functions of said at least first and second types of detector cells.

According to some embodiments, the detector array is adapted for collecting image data using said at least first and second types of detector arrays simultaneously.

According to some embodiments, the detector array comprises said comprises at least first and second types of detector cells arranged in an interlaced order within a common plane of the detector array, such that image data generated by said detector array comprises at least first and second image portions of a common field of view and associated with said first and second different spectral response functions

Additionally, or alternatively, according to some embodiments, the detector array may comprise three or more different types of detector cells comprising at least said first and second types of detector cells and at least a third type of detector cells. The three or more types of detector cells may comprise detector cells having spectral response functions having spectral peaks corresponding to red, green and blue light.

According to some embodiments, the light source unit may be adapted, or configured, for emitting at least first and second beams of optical illumination corresponding to said at least first and second discrete wavelength ranges toward at least a portion of field of view of the imaging unit.

According to some embodiments, the at least first and second discrete wavelength ranges are spectrally non-overlapping.

According to some embodiments, the first and second spectral peaks may correspond to blue and orange-red illumination colors.

According to some embodiments, the light source unit may comprise at least first and second light sources configured for respectively emitting said light comprising said at least first and second discrete wavelength ranges. The first and second light sources may be narrow band light sources. Additionally, or alternatively, the first and second light sources may be configured to emit light having defined color.

According to some embodiments, the two or more discrete wavelengths of illumination may comprise illumination within ranges 400-570 nm and 580-770 nm. The first and second different wavelengths may correspond to wavelengths within the ranges 400-480 nm and 580-700 nm. Preferably, the first and second different wavelengths may correspond to wavelengths within the ranges 405-420 nm and 630-670 nm. Alternatively, the first and second different wavelengths may correspond to wavelengths within the ranges 410-420 nm and 640-660 nm.

According to some embodiments, the imaging unit may further comprise a wavelength blocking filter configured for blocking selected input radiation. The blocking filter may comprise an infrared blocking filter configured for filtering out infrared illumination.

According to some embodiments, the light source unit may be adapted, or configured, to provide the illumination within said at least first and second discrete wavelength ranges simultaneously, and at least partially concurrently with operation of said imaging unit for acquisition of image data, such that exposure time of the imaging unit at least partially overlaps with a time period of said illumination.

The system is associated with (i.e. comprises or is connectable to) a processing unit adapted for receiving image data from said detector array during image acquisition by said imaging unit of light collected from a region of interest subjected to said illumination, and for processing said image data to extract therefrom first and second image data pieces corresponding to collected light in the at least two different wavelength ranges and generate output data indicative of an enhanced image of the region of interest (e.g. biological tissue). Such output data may be indicative of an image map based on a relation between selected functions of the at least first and second image data pieces, providing enhancement to contrast of a selected portion of the region of interest (e.g. blood vessels) over surrounding portions of said region of interest (e.g. tissue region).

The processing unit may comprise an intensity calibration module adapted for operating in calibration mode defining an intensity calibration condition according to which intensity of illumination, generated by said light source unit in at least first and second discrete wavelength ranges, provides for obtaining substantially similar intensity response by said first and second types of detector cells.

The processing unit may be adapted for automatically operating said intensity calibration module, and upon determining that said intensity of illumination satisfies the calibration condition, operating the detector array for acquiring image data and processing said first and second image data pieces to generate output data.

According to some embodiments, the intensity calibration module may be adapted for operating said light source unit and imaging unit for collecting image data under illumination of said at least first and second discrete wavelength ranges, determining saturation level for said first and second types of detector cells and calibrating illumination intensity for said at least first and second discrete wavelength ranges in accordance with the selected saturation level.

According to some embodiments, the processing unit may be adapted for operating said light source unit and said imaging unit for illuminating a field of view and collecting image data simultaneously.

According to some embodiments, the processing unit may be adapted for operating the light source unit in continuous illumination mode and/or in flash illumination mode

According to some embodiments, the imaging unit may further comprise an optical lens arrangement adapted for selectively varying focusing distance for imaging in accordance with data on light collected by selected one of said at least first and second types of detector cells individually.

The imaging unit may be adapted for determining the focusing condition in accordance with light of said first or second wavelength ranges selectively.

According to some embodiments, the system described herein may be configured for obtaining enhanced image data of biological tissue. For example, the system may be configured for obtaining enhanced image data associated with blood vessels of a tissue region. Such an enhanced image, with proper selection of wavelengths for illumination and collection (e.g. types of detector cells and corresponding maximal response wavelength) may enable detection of blood oxygenation levels. According to some embodiments, the system described herein may be adapted or configured for obtaining enhanced image data associated with blood vessels in at least one of retina and sclera of a patient's eye.

According to one other broad aspect, the present invention provides a method for acquiring image of biological tissue, the method comprising: providing image data, which corresponds to a light response of a region of interest to illumination of at least first and second different wavelength ranges, and is collected by a detector array comprising at least first and second different types of detector cells having corresponding first and second different spectral response functions defining respectively first and second spectral peaks aligned with said at least first and second different wavelength ranges, respectively; processing said image data by extracting therefrom at least first and second image data pieces associated with the collected light response by said at least first and second different types of detector cells, and generating output data indicative of an image map in accordance with a relation between said at least first and second image data pieces, said image map thereby providing enhanced contrast image of the region of interest.

The enhanced contrast image of the region of interest is characterized in enhancement of contrast of a selected portion of the region of interest over surrounding portions of said region of interest being imaged

According to some embodiments, said image data is collected during an exposure time of said detector array at least partially overlaps with a time period of said illumination.

According to some embodiments, said image data corresponds to simultaneous illumination of the region of interest by said at least first and second different wavelength ranges.

According to some embodiments, the at least first and second wavelength ranges are spectrally non-overlapping or at least partially spectrally non-overlapping.

According to some embodiments, the method may further comprise determining focusing state of an optical arrangement for collection of said image data in accordance with collection of light of one of said at least first and second different wavelength ranges.

According to some embodiments, the method may further comprise selectively determining the focusing state of an optical arrangement for collection of said image data in accordance with selected type of detector cells adapted for collection of light.

According to some embodiments, the method may further comprise: determining an initial focusing state providing sharp image (relatively), varying a focusing state by a selected amount to provide blurred image (relatively), adjusting (returning) a focusing state toward said initial focusing state in a plurality of small focus steps, for each of said plurality of small focus steps determining a focusing level indicative of sharpness of the collected image is said one of said at least first and second wavelength ranges, and determining focusing state in accordance with focus step having maximal focusing level.

The plurality of small focus steps may pass (e.g. overshoot) the initial focusing state to its other focal side.

According to yet another broad aspect, the present invention provides a method for use in imaging a biological tissue, the method comprising providing image data, which corresponds to a light response of the biological tissue to illumination of at least first and second wavelength ranges and is collected using a detector array comprising at least first and second types of detector cells characterized by corresponding first and second spectral response functions having respectively first and second spectral peaks at different first and second wavelengths aligned with said first and second wavelength ranges of the illumination.

The first and second wavelength ranges may be non-overlapping.

According to some embodiments, the method may further comprise processing the image data collected by the detector array by extracting at least first and second image data pieces associated with image portions collected by said at least first and second types of detector cells, and determining an enhanced image of the biological tissue by determining a relation between said first and second image data portions.

According to some embodiments, the method may further comprise calibrating illumination intensity for said first and second wavelength ranges; said calibration comprising determining initial intensity level for illumination with said first and second wavelength ranges, collecting first image data, determining saturation levels for detector cells of said first and second types of detector cells and adjusting intensity level for illumination with one or more of said first and second wavelength ranges to provide predetermined saturation levels.

Said calibrating illumination intensity may comprise iteratively repeating said calibration until reaching at least one of said predetermined saturation levels and predetermined iterative cycles.

Said saturation levels may be determined by intensity histogram of detector cells of the same type.

According to some embodiments, said predetermined saturation level may be associated with the difference between intensity histogram of the first and second types of detector cells being within predetermined limits.

According to some embodiments, said calibrating illumination intensity for said first and second wavelength ranges comprises determining one or more contrast measures for at least first and second image portions and determining variation in illumination level for at least one of the first and second wavelength ranges to optimize contrast of the first and second image portions.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates schematically a system for use in angiographic imaging according to some embodiments of the present invention;

FIG. 2 exemplifies typical spectral response functions of an RGB optical detector array;

FIG. 3 shows a flow chart exemplifying a technique for providing image with enhanced contrast according to some embodiments of the invention;

FIG. 4 shows a flow chart exemplifying a technique for adjusting illumination calibration according to some embodiments of the invention; and

FIG. 5 exemplifies a block diagram configuration of a processing unit according to some embodiments of the invention.

### DETAILED DESCRIPTION OF EMBODIMENTS

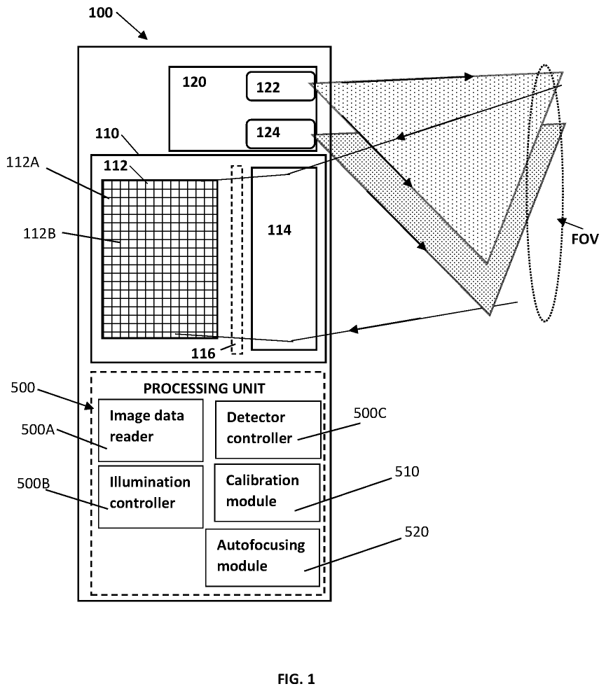

As indicated above, the present technique provides a system and corresponding method for use in enhanced angiographic imaging of biological tissue. Reference is made to FIG. 1 schematically illustrating system 100 including an imaging unit 110 and a light source unit 120.

The system 100 is associated with (i.e. includes or is connectable to) a processing unit 500 configured for providing operational data for operating the imaging unit 110 and light source unit 120. In some embodiments, the processing unit 500 is also configured for processing image data, generated by the imaging unit, as described in detail below.

The imaging unit 110 includes a detector array 112 and an optional corresponding optical arrangement 114 configured and positioned for defining a selected field of view FOV for light collection therefrom onto the detector array 112 during an imaging session. The detector array 112 includes a plurality of detector cells, e.g. typically arranged in a two-dimensional array, including detector cells of two or more types having different spectral response functions defining respectively first and second spectral peaks, two such types of detector cells, generally at 112A and 112B, being exemplified in the non-limiting example of FIG. 1. The detector cells of different types may be distributed in any suitable in a selected arrangement.

More specifically, the detector array 112 includes detector cells of different response functions. This can be achieved by either using the detector cells of the same type equipped with suitable filter(s), or using the detector cells of different types, i.e. having different spectral sensitivity. The detector cells of different response functions are thus configured for collecting light components of selected different wavelengths (wavelength ranges) in accordance with their spectral response functions. This allows the detector array 112 to collect color image data by separating the collected light to spectral portions.

For example, typical color detector arrays include three types of detector cells (generally using monochromatic detector cells and Bayer filter array) configured for collection of light of three different colors such as primary colors RGB, i.e. red, green and blue. The present technique may utilize such detector configuration and may also use a detector configuration having an arrangement of detector cells of two or more different types.

For simplicity, the detector array 112 is described herein as including first and second different types of detector cells 112A and 112B having corresponding first and second different spectral response functions. It should however be understood that the principles of the present invention are not limited to this specific example, as well as not limited to any specific number n≥2 of the different types of the detector cells.

As also shown in FIG. 1, the imaging unit may also include spectral blocking filter 116 configured for blocking collection of selected spectral range.

The light source unit 120 is configured to provide illumination having at least two discrete and different wavelengths (or wavelength ranges) directed toward a region of interest within the field of view FOV of the imaging unit. The light source unit 120 may typically include two or more light sources 122 and 124 (e.g. LED light sources) configured for emitting light of at least first and second different wavelength ranges selected in accordance with spectral response function of the first and second types of detector cells. For example, the light source(s) may emit two or more beams, including beams including light beam(s) of the first wavelength range and light beam(s) of the second wavelength range.

The light source unit 120 is preferably configured to provide narrow band illumination, such that the at least two wavelength ranges do not overlap in spectral bandwidth, providing illumination with light of two different colors. In some configurations, the at least two wavelengths of illumination correspond to at least two wavelength ranges having partial overlap, while being aligned with spectral peaks in the response functions of the detector cells of different types. More specifically, the at least two wavelengths of illumination are distinguishable when illumination thereof is collected by the detector cells of the detector array 112.

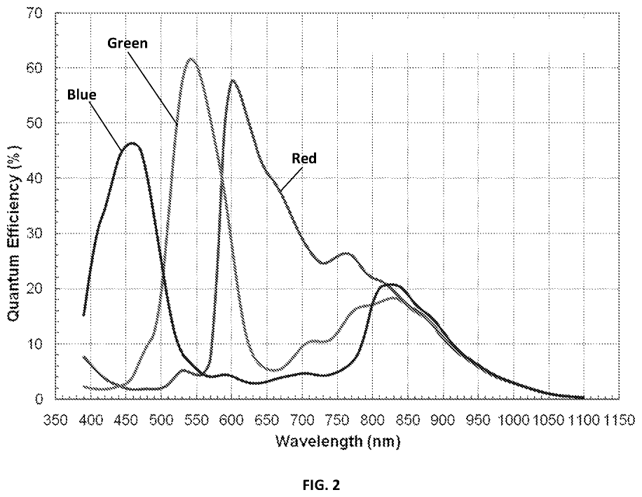

According to the present technique, the at least two wavelengths (wavelength ranges) of illumination are selected in accordance with spectral response functions of the first and second types of detector cells of the detector array 112. FIG. 2 shows spectral response functions of an exemplary color detector array having RGB color configuration (e.g. using a Bayer filter). The figure shows spectral response function for detectors cells configured for collecting Blue light, for detector cells configured for collecting Green light, and for detector cells configured for collecting Red light. As shown, each spectral response function has a spectral peak for a specific wavelength, different from those of the other response functions. In this specific and non-limiting example, the spectral peak for Blue light is at wavelength about 465 nm, the spectral peak for Green light is at wavelength about 540 nm and the spectral peak for Red light is at wavelength about 600 nm.

As indicated above, the first and second wavelength ranges used for illumination are selected in accordance with wavelengths of the first and second spectral peaks of the respective first and second spectral response functions corresponding to the first and second types of detector cells. More specifically, according to some embodiments of the present technique, the first and second types of detector cells include detector cells configured for collection of blue light components and for collection of red-light components. In accordance with the example of FIG. 2, the light source unit 120 may generally include light source 122 configured for emitting light at a wavelength range around the spectral peak of the “blue type” detector cells, and light source 124 configured for emitting light at a wavelength range around the spectral peak of the “red-type” detector cells. Accordingly, the light source unit 120 provides illumination with a set of at least first and second discrete wavelength ranges spectrally aligned with the spectral peaks of the response functions of at least the first and second types of detector cells.

Light source unit 120 may include two or more light sources 122 and 124 configured for providing illumination in the discrete wavelength ranges aligned with the spectral peaks of two or more types of detector cells of the detector array 112. More specifically, for use with typical detector array 112 configured for collecting light in three different wavelength ranges by respective three different types of detector cells, the light source unit 120 may include two or three different light sources for emitting light in two or three different wavelength ranges (being non-overlapping). For example, a typical RGB detector may have detector cells having maximal response for wavelengths of 450 nm (blue), 550 nm (green) and 650 nm (Red). For use with such a detector array, the light source unit may include light sources (e.g. LED light sources) configured for emitting light in narrow bands around at least two from wavelengths of 450 nm, 550 nm and 650 nm.

In some examples, the light source unit 120 is configured to provide two or more discrete wavelength ranges of illumination including a first wavelength range being a relatively narrow band within the range of 400-570 nm and a second wavelength range being a relatively narrow band within the range of 580-770 nm. The first and second different wavelength ranges may correspond to wavelength ranges having a bandwidth of 10-50 nm within the ranges 400-480 nm and 580-700 nm.

In some examples, the first and second different wavelength ranges may include light within the ranges of 405-420 nm and 630-670 nm or within the ranges 410-420 nm and 640-660 nm.

Additionally, in some configurations, the imaging unit 110 may also include a spectral blocking filter 116 configured for blocking collection of selected spectral range. For example, the imaging unit 110 may utilize an infrared blocking filter configured for filtering out infrared illumination. As shown in FIG. 2, some RGB detector cells may have similar spectral response functions with respect to incident light at wavelengths over 800 nm. Accordingly, the spectral blocking filter 116 may be used for reducing an overlap in detection of light between the types of detector cells and thus for increasing the signal to noise ratio.

Turning back to FIG. 1, system 100 may be associated with a processing unit 500. The processing unit 500 is generally connected (by wires or wireless data communication) to the imaging unit 110 and to the light source unit 120. The processing unit 500 includes an illumination controller 500B, a detector controller 500C, and an image data reader 500A. The processing unit 500 is thus capable of for providing operational data (operation commands) to the light source unit and imaging unit and for receiving image data from the detector array 112. The processing unit 500 may also include one or more processors and memory utilities. For example, the image data reader 500A may be and be adapted for processing and analyzing the image data from the detector array 112 to generate output data in the form of enhanced angiographic image. As also shown in the figure, the system 100 preferably also includes a calibration module 510, the purpose and operation of which will be described further below. As further shown in the figure, and will be described further below, the processing unit 500 may include an autofocusing module 520.

The illumination controller 500B of the processing unit 500 may operate the light source unit 120 to emit light having the first and second wavelength ranges (e.g. using light sources 122 and 124) and illuminate a region of interest within the field of view FOV of the detector. The detector controller 500C of the processing unit 500 is configured for operating the imaging unit 110 to perform one or more imaging sessions for collection of image data during a time period (exposure time) at least partially overlapping with the illumination time period. The light source unit 120 may be operated in flash mode, i.e. providing high intensity illumination for a short time, or in continuous illumination mode to provide illumination for a period substantially longer with respect to exposure time of the detector array 112. The detector array 112 is operated for collecting light components from the field of view FOV and generating corresponding image data associated with at least first and second wavelength ranges of light arriving from the field of view FOV.

The use of at least first and second wavelength ranges is based on the inventors' understanding that a relation between collected image data in at least two different wavelength ranges enables contrast enhancement for imaging of blood vessels over background of biological tissue. More specifically, using at least two image portions of a tissue sample, where one image is collected at a first (e.g. blue) wavelength range and another image is collected at a second (e.g. red) wavelength range, allows determining an image map based on a relation (e.g. ratio) between selected functions of the at least two image portions. Such an image map provides enhancement to contrast of blood vessels over surrounding biological tissue. To this end, the present technique utilizes processing image data received from the detector array 112 for extracting at least two image portions associated with image collected by the first type of detector cells (e.g. blue detector cells) and image portions associated with image collected by the second type of detector cells (e.g. red detector calls). For example, the detector array 112 generates image data in the form of RGB image (e.g. bitmap or compressed color image), and processing of the image data may comprise extraction of Red image portion and of Blue image portion of the image data and determination of a contrast enhanced image corresponding to a selected ratio between the red and blue image portions.

Thus, the present invention utilizes image portions collected by a common detector array, collected in a common instance of image acquisition, to avoid the need for registration between pixels of different images.

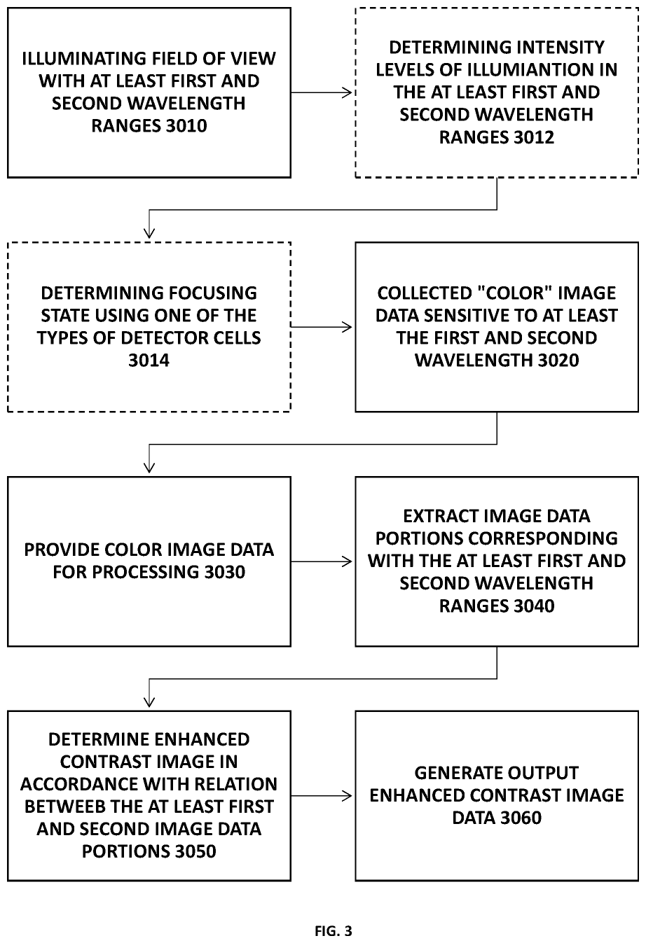

In this connection, reference is made to FIG. 3 exemplifying operation of the present technique in a flow chart. As shown, the present technique utilizes illuminating a field of view with at least first and second wavelength ranges 3010. The first and second wavelength ranges are selected as described above to be generally discrete and be spectrally aligned with peaks of spectral response functions of the first and second types of detector cells of an imaging unit. In combination with image collection, the technique may utilize determining intensity levels of the illumination with the selected wavelength ranges 3012 as described in more details further below and may include determining focusing state based on selected one of the types of detector cells 3014. The order of steps 3012 and 3014 is not significant and therefore it can be interchangeable.

Under this illumination condition, the technique includes collecting image data using a detector array having the at least first and second types of detector cells 3020. This image data may generally be a color image of the field of view, while being affected by illumination condition of the first and second wavelength ranges. In some embodiments, one or more so-collected image data pieces may be used for further processing 3030. The processing may include extracting at least first and second image portions 3040 associated with the first and second types of detector cells. For example, using an RGB color image detector, an image data piece may be formed by three-pixel maps indicating the intensity of light collected by the three types of detector cells. More specifically, the three-pixel maps may indicate the intensity of light collected by the red detector cells, green detector cells and blue detector cells. It should be noted that certain actions indicated herein with reference to FIG. 3 may be performed simultaneously and/or in varying order. Further, as illustrated in FIG. 3, certain actions are marked in dashed line to illustrate that these actions may be optional and may provide further improvement to the technique, but may also be omitted depending on the specific configuration.

The processing includes determining a map of relations between the at least first and second image portions for generating an enhanced contrast image 3050, and generating output data 3060 indicative of the enhanced contrast image. For example, the enhanced contrast image may be determined in accordance with a ratio between detected light intensity by the detector cells of different types (e.g. red and blue types) for each pixel.

For example, the output image may be in the form of:

$$Im ( i , j ) = \frac{{( {Im}^{R} ( i , j ) )}^{n}}{{( {Im}^{B} ( i , j ) )}^{m}}$$

where Im(i,j) is the enhanced contrast image pixel (i,j), Im^{R}(i,j) is pixel (i,j) of the red image portion and Im^{B}(i,j) is pixel (i,j) of the blue image portion, n and m are real numbers. It should be noted that in some configurations the enhanced contrast image may be determined in accordance with the relation between green and blue image portions, or red and green image portion. In some additional examples, the output image may be in the form of:

$$Im ( i , j ) = \frac{{α ( {Im}^{1} ( i , j ) )}^{n} + {β ( {Im}^{3} ( i , j ) )}^{l}}{{γ ( {Im}^{2} ( i , j ) )}^{m}}$$

Where Im¹(i,j), Im²(i,j) and Im³(i,j) relate to pixel (i,j), in the red, green or blue image portions and α, β and γ are selected coefficients. In some configurations, the summation of pixels may be performed in the readout stage to simplify processing.

The present technique utilizes illumination with at least two different and non-overlapping wavelength ranges and collection of image data using a detector array having at least two different types of detector cells (detector cells sensitive to different wavelength ranges) for providing image portions in a common image acquisition using common optics. This enables processing of the image data while avoiding the need to apply registration process where pixels of one image portion need to be aligned with pixels of one other image portion. This is advantageous for obtaining angiographic images of biological tissues that tend to move at high speed. For example, angiographic images of tissue in a patient's eye may typically require high speed image acquisition for compensating high speed eye movement.

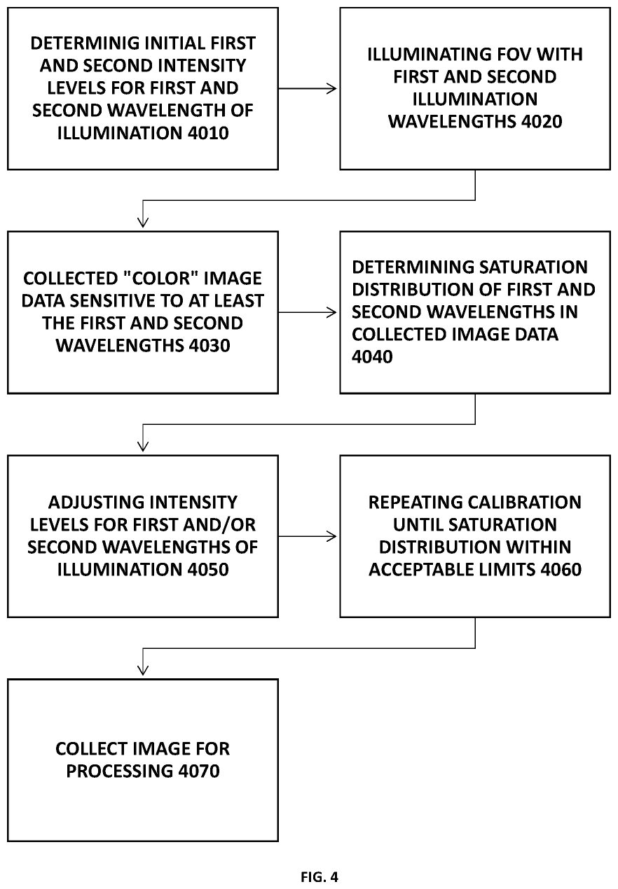

To enable further enhancing of image contrast, allowing improved angiography, the present technique may utilize an illumination calibration process. More specifically, the calibration procedure is aimed at adjusting illumination intensity of different wavelength ranges to sensitivity of the detector cells of different types (i.e. having different spectral response functions). To this end, the system of the present the present invention includes a calibration module (510 in FIG. 1) configured and operable to perform an illumination calibration process. This is exemplified in FIG. 4.

Generally, initial first and second intensity levels are determined (step 4010) for operating the light source to provide illumination with the at least first and second wavelength ranges. Such initial intensity levels may be similar or different for the different wavelengths of illumination and may be predetermined or selected by an operator. An imaging session is performed, and an image is acquired by illuminating the field of view with the different wavelength ranges of selected intensity levels (step 4020) and collecting light response of the illuminated region of interest by a detector array having two or more types of detector cells having different spectral response functions (step 4030), as described above. The so-detected light response provides collected “color” image data sensitive to at least the first and second wavelengths.

The image data is processed by extracting therefrom image portions corresponding to the two different types of detector cells and determining intensity levels (saturation levels) of the collected image portions 4040.

For example, using 8-bit digital detector cells, the intensity levels of the acquired image portions may range between 0 and 255. A high number of pixels measuring intensity at 255 may indicate saturation of the detector, while if there are no pixels measuring at high intensities (e.g. no pixels measuring above 250) the range of detection is limited.

It should be noted that the light intensity may be determined by any known suitable technique e.g. using wavelets and determining amplitude at high spatial frequencies, using analysis of contrast of the first and second image portions, etc.

Generally, to provide high quality improved contrast, the intensity levels in the different image portions are preferably substantially similar, while utilizing the dynamic range of the detector cells.

In accordance with the intensity levels of the image portions corresponding to the two different types of detector cells, the calibration module 510 operates together with the illumination controller 500B for adjusting intensity of illumination in one or more of the wavelength ranges (step 4050), and the calibration procedure (steps 4020, 4030 and 4040) is repeated (step 4060) until a condition of substantially similar intensity levels in the different image portions is provided. Generally, when intensity levels of detected light in the at least two different image portions are sufficiently close, the so-acquired image may be used for processing (step 4070).

Adjusting the illumination intensity levels can further enhance contrast for angiographic imaging by utilizing the full dynamic range of the detector cells. Considering imaging of a region of interest on a subject's body, the above-described calibration of illumination intensities enables enhancing detection of blood vessels within the image data of the region of interest in accordance with variation in reflective properties to different wavelength ranges.

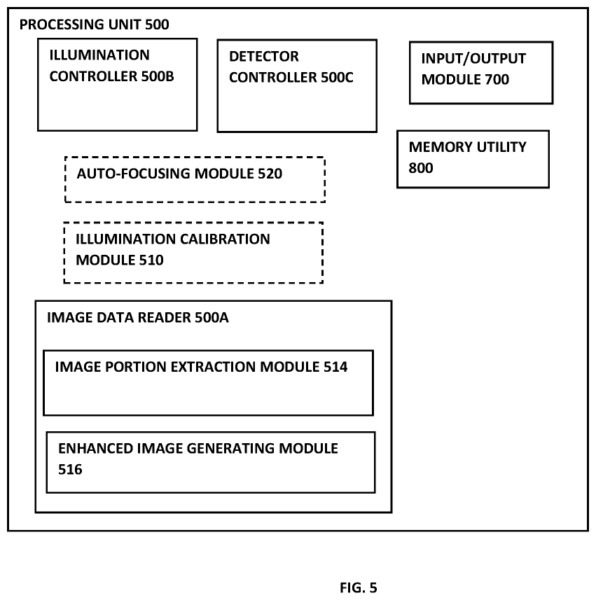

Reference is made to FIG. 5 illustrating, by way of a block diagram, a specific but not limiting example of the functional utilities of the processing unit 500 according to some embodiments of the present invention. The processing unit 500 is generally configured as a computing unit including data input/output module 700 and memory utility 800, and includes the illumination and detector controllers 500B and 500C, and image data reader 500A. The illumination and detector controllers 500B and 500C are configured and operable for generating and directing operational commands to the light source unit 120 and the imaging unit 110 respectively.

The image data reader 500A is configured and operable for processing and analyzing image data provided by the detector array. To this end, the image data reader 500A includes an image portion extraction module 514 configured for receiving polychromatic image data (e.g. RGB image data) from the detector array and extracting image portions associated with the different two or more types of detector cells; and an enhanced image generating module 516 configured for using two of more image portions received from the mage portion extraction module 514 and predetermined or selected parameters pre-stored in the memory utility 800 for determining an enhanced contrast image of a region of interest in the field of view.

The processing unit 500 may also include the autofocusing module 520 and/or the illumination calibration module 510. The operation of the illumination calibration module 510 is described above. It should be understood that the illumination calibration module 630 may be a component of the illumination controller 510.

The autofocusing module 520 is configured and operable for tuning the focusing state of the imaging unit based on one or more of the image portions extracted by the extraction module from the detected image data. Generally, the autofocusing module 520 may operate for determining optimal focusing state of the optical lens arrangement (114 in FIG. 1) associated with the imaging unit 110. The autofocusing module 520 utilizes data indicative of one or more of the extracted image portions for determining a focusing level of the optical lens arrangement. Thus, the autofocusing module 520 is operated for tuning the focus of the optical lens arrangement 114 in accordance with the image portions of one or more colors (wavelength ranges), rather than using generally monochromatic image data.

The above technique allows to utilize a difference in penetration depths of light of the respective wavelength ranges into different tissue portions (biological tissues) with the region of interest being imaged, for imaging the tissue portions (blood vessels) at selected depths.

More specifically, in the example of typical RGB imaging, i.e. using a standard industrial color camera with three color channels R (Red), G (Green) and B (Blue) with peak responses at 650 nm, 550 nm and 450 nm correspondingly, and providing illumination in at least two of the peak response levels (e.g. illumination in 650 nm and 450 nm, and possible also in 550 nm), light components of different wavelengths have slightly different penetration depths into biological tissue. More specifically, light of a wavelength around 450 nm may have penetration depth within the range of 200-400 micrometers, while light components at wavelength of 650 nm may penetrate deeper into the tissue, and provide penetration depth of 500 micrometer or more. Accordingly, depending on depth of focus of the optical lens arrangement, determining focusing based on input of blue detector cells may result in imaging of a plane at 200-400 micrometers penetration depth into the tissue, and determining focusing using red detector cells may provide imaging of deeper layers of the tissue (generally 500-1000 micrometers). It should be noted that selection of the wavelength used for illumination is performed in accordance with the peak response of the detector cells, and may also be selected in accordance with variation in reflection properties of the tissue being imaged.

The autofocusing module 520 may utilize any known suitable technique for determining the level of focusing. For example, in some configurations, the autofocusing module 520 may be configured for determining contrast between sub-groups of detector cells of a selected type, selected from one or more regions of a collected image. Contrast between neighboring pixels may provide indication to sharpness of the image. Additional autofocusing technique may utilize phase detection. In these configurations, light components arriving from a common location in the inspected tissue (sample) and passing through different regions of the optical lens arrangement are compared at the detector plane. When the optical arrangement is properly focused, such light components overlap at the detector plane, while if the optical lens arrangement is out of focus, two or more not overlapping image regions may be identified.

As indicated, the focusing state/level is preferably determined using selected one type of detector cells. However, an initial focusing level may be determined based on monochromatic imaging or combination of the different wavelength ranges. This configuration of the focusing detection, combined with illumination of two or more different wavelength ranges aligned with maximal response to the different types of detector cells, enables focusing on object planes being in selected penetration depth(s) of light of selected wavelength range(s) into the biological tissue.

To provide suitable focusing, while enabling detection of the differences between the penetrations depths into the tissue, the autofocusing module 520 may be adapted to determine focusing state/level at one of more object planes, and the focusing state may be determined using detector cells of the selected type.

Generally, this technique may include: determining initial focusing state providing sharp image, varying focusing state by a selected amount to provide blurred image, returning focusing state toward said initial focusing state in a plurality of small focus steps, for each of said plurality of small focus steps determining a focusing state indicative of sharpness of the collected image is said one of said at least first and second wavelength ranges, and determining focusing state in accordance with focus step having maximal focusing state.

In some examples, the plurality of small focus steps pass said initial focusing state to its other focal side. More specifically, if the first diversion from initial focusing state is directed to focusing on object plane that is further from the imaging unit, the technique may utilize progress in small focus steps and overshoot toward defocusing for object plane located closer with respect to the imaging unit and vice versa. It should be noted that, generally, the present technique may utilize selection of preferred penetration depth for which optimal focusing is desired. In accordance with the preferred penetration depth, the wavelength, or type of detector cells, used for autofocusing is selected based on the penetration depth of the corresponding illumination wavelength.

Thus, the present technique provides for novel imaging technique enabling improved and enhanced contrast imaging. This technique may enable improved imaging of biological tissues allowing angiographic imaging, by enabling detection of blood vessel from optical imaging that does not require administration of contrast material. The technique of the present invention may be advantageously used for angiographic imaging of eye regions, such as retina and sclera where rapid eye movement does not allow collection of separated images.

1. A system, comprising:
a light source unit;
at least one imaging unit comprising a detector array, wherein the detector array comprises at least first and second types of detector cells having corresponding first and second different spectral response functions defining respectively first and second spectral peaks; and
a light source unit configured for emitting light forming illumination including at least first and second discrete wavelength ranges, said at least first and second discrete wavelength ranges being aligned with said first and second spectral peaks of said first and second types of detector cells.

2. The system of claim 1, wherein said light source unit is adapted to provide the illumination within said at least first and second discrete wavelength ranges simultaneously, and at least partially concurrently with operation of said at least one imaging unit for acquisition of image data, such that exposure time of the at least one imaging unit at least partially overlaps with a time period of said illumination.

3. The system of claim 1, wherein said detector array is adapted for collecting image data using said at least first and second types of detector arrays simultaneously.

4. The system of claim 1, wherein said detector array comprises wavelength selective filter array filtering collected light and defining at least a portion of said first and second spectral response functions of said at least first and second types of detector cells.

5. The system of claim 1, wherein said detector array comprises said at least first and second types of detector cells arranged in an interlaced order within a common plane of the detector array, such that image data generated by said detector array comprises at least first and second image portions of a common field of view and associated with said first and second different spectral response functions.

6. The system of claim 1, wherein said detector array comprises three or more different types of detector cells comprising at least said first and second types of detector cells and at least a third type of detector cells selected from detector cell having spectral response functions having spectral peak corresponding to red, green and blue light.

7. The system of claim 1, wherein said at least first and second discrete wavelength ranges in the emitted light are spectrally substantially non-overlapping.

8. The system of claim 1, wherein said at least one imaging unit further comprises a wavelength blocking filter configured for blocking selected input radiation.

9. The system of claim 1, wherein said light source unit comprises at least first and second light sources configured for respectively emitting said light formed of said at least first and second discrete wavelength ranges.

10. The system of claim 1, further comprising a processing unit adapted for receiving image data from said detector array associated with image acquisition by said imaging unit of light collected from a region of interest subjected to said illumination, and for processing said image data to extract therefrom first and second image data pieces corresponding to collected light in the at least two different wavelength ranges and generate output data indicative of an enhanced image of the region of interest; said output data is indicative of an image map based on a relation between selected functions of the at least first and second image data pieces, providing enhancement to contrast of a selected portion of the region of interest over surrounding portions of said region of interest.

11. The system of claim 10, wherein said processing unit comprises an intensity calibration module adapted for operating in calibration mode defining an intensity calibration condition according to which intensity of illumination, generated by said light source unit in at least first and second discrete wavelength ranges, provides for obtaining substantially similar intensity response by said first and second types of detector cells.

12. The system of claim 11, wherein said intensity calibration module is adapted for operating said light source unit and said imaging unit for collecting image data under illumination of said at least first and second discrete wavelength ranges, determining saturation level for said first and second types of detector cells, and calibrating the intensity of illumination for said at least first and second discrete wavelength ranges in accordance with selected saturation level.

13. The system of claim 1, wherein said at least one imaging unit further comprises an optical lens arrangement adapted for selectively varying a focusing state for imaging in accordance with data on light collected by selected one of said at least first and second types of detector cells individually; and wherein said imaging unit is adapted for determining the focusing state in accordance with light of said first or second wavelength ranges selectively.

14. The system of claim 1, wherein said detector array is selected in accordance with said first and second spectral peaks for obtaining enhanced image data associated with blood vessels of a biological tissue in the region of interest.

15. The system of claim 14, configured for obtaining enhanced image data associated with blood vessels in at least one of retina and sclera of a patient's eye.

16. A method for acquiring image of biological tissue, the method comprising:
providing image data, which corresponds to a light response of a region of interest to illumination of at least first and second different wavelength ranges, and is collected by a detector array comprising at least first and second different types of detector cells having corresponding first and second different spectral response functions defining respectively first and second spectral peaks aligned with said at least first and second different wavelength ranges, respectively;
processing said image data by extracting therefrom at least first and second image data pieces associated with the collected light response by said at least first and second different types of detector cells, and
generating output data indicative of an image map in accordance with a relation between said at least first and second image data pieces, said image map thereby providing enhanced contrast image of the region of interest.

17. The method of claim 16, wherein said image data is collected during an exposure time of said detector array at least partially overlaps with a time period of said illumination, and wherein said image data corresponds to simultaneous illumination of the region of interest by said at least first and second different wavelength ranges.

18. The method of claim 16, further comprising determining a focusing state of an optical arrangement for collection of said image data in accordance with collection of light of one of said at least first and second different wavelength ranges.

19. The method of claim 16, further comprising: determining an initial focusing state providing a relatively sharp image, varying a focusing state by a selected amount to provide a relatively blurred image, returning a focusing state toward said initial focusing state in a plurality of small focus steps, for each of said plurality of small focus steps determining a focusing level indicative of sharpness of the collected image is at least one of said at least first and second wavelength ranges, and determining the focusing state in accordance with a focus step having a maximal value of the focusing level.

20. The method of claim 16, further comprising calibrating illumination intensity for said first and second wavelength ranges; said calibration comprising determining initial intensity level for illumination with said first and second wavelength ranges, collecting first image data, determining saturation levels for detector cells of said first and second types of detector cells, and adjusting intensity level for illumination with one or more of said first and second wavelength ranges to provide predetermined saturation levels.
